(19) United States
(12) Patent Application Publication
(10) Pub. No.: US 2024/0180818 A1
(43) Pub. Date: Jun. 6, 2024

(54) SKIN CARE FORMULATIONS WITH HYDROPHILIC ACTIVE INGREDIENT

(71) Applicant: Metselaar Amsterdam Beheer B.V., Naarden (NL)

(72) Inventor: Josbert Maarten Metselaar, Naarden (NL)

(21) Appl. No.: 18/553,493

(22) PCT Filed: Mar. 31, 2022
(86) PCT No.: PCT/NL2022/050181

§ 371 Date: Sep. 29, 2023

(30) Foreign Application Priority Data
Mar. 31, 2021 (NL) 2027878

(51) Int. Cl. A61K 9/00; A61K 8/06; A61K 9/107; A61K 47/14; A61K 47/44; A61Q 19/00

(52) U.S. Cl. CPC A61K 9/0014; A61K 8/06; A61K 9/107; A61K 47/14; A61K 47/44; A61K 2800/596; A61Q 19/00

(57) ABSTRACT

This disclosure relates to a composition comprising a continuous hydrophobic phase, a hydrophilic phase dispersed in the hydrophobic phase, a hydrophilic active ingredient dissolved in the hydrophilic phase, and a stabilizing component comprising 4-20 wt % or 4-7.9 wt % of an emulsifier having a hydrophilic-lipophilic balance (HLB) value of at least 10, and 80-96 wt % or 92.1-96 wt % of an emulsifier having an HLB value of less than 10, wherein the continuous hydrophobic phase optionally comprises a solid lipid component comprising 50-100 wt % of one or more high melting point triglycerides and 0-50 wt % of one or more waxes and optionally wherein the weight ratio of the stabilizing component to the hydrophilic active ingredient is less than 10:1.

### CROSS REFERENCE TO RELATED APPLICATIONS

This application is a national phase entry under 35 U.S.C. § 371 of International Patent Application PCT/NL2022/050181, filed Mar. 31, 2022, designating the United States of America and published as International Patent Publication WO 2022/211632 A1 on Oct. 6, 2022, which claims the benefit under Article 8 of the Patent Cooperation Treaty to Dutch Patent Application Serial No. 2027878, filed Mar. 31, 2021.

### TECHNICAL FIELD

This disclosure relates to the field of pharmaceutical or cosmetic composition, in particular formulations for topical administration to the skin. The compositions comprise a continuous hydrophobic phase and a hydrophilic phase dispersed therein. This disclosure further relates to methods for the preparation of such compositions and uses thereof.

### BACKGROUND

Many biologically active compounds (e.g., pharmaceutically and/or cosmetically active compounds) are highly hydrophilic. Formulating the biologically active hydrophilic compounds into a formulation that can be used for application on the skin is most straightforward when these compounds are dissolved in a continuous phase that is based on water. However, there are major drawbacks to the use of water as a continuous phase in skincare products, which include the need for preservatives (which can entail safety issues and affect the human skin's own microbiome), the possibility to dehydrate the skin, and most importantly, the fact that many biologically active hydrophilic compounds are inherently chemically unstable in an aqueous environment.

The formulation of biologically active hydrophilic compounds in water-free (anhydrous) skincare products on the other hand presents considerable challenges. Such formulations may be based on hydrophilic organic solvents as the continuous phase in which the active compounds can be dissolved, for example, propylene glycol or poly(ethylene glycol) 400 or 800, but these formulations do not provide an acceptable skin feel and score low on a consumer perception scale. The best water-free/anhydrous skincare products from a consumer perspective are based on a continuous phase composed of oils, fats and waxes from natural or mineral sources, which are essentially hydrophobic. Clearly, such continuous phases do not allow for the dissolution of hydrophilic compounds and formulations of such actives in oils, fats and waxes can only be successfully achieved by dispersion. However, dispersed formulations often have poor physical stability, and tend to phase separate or lead to precipitation, resulting in a poor shelf stability and inconsistent performance.

WO 2016/118907 describes formulations that include a continuous hydrophobic phase, a hydrophilic phase in which a hydrophilic compound is dissolved, and a stabilizing component that comprises a minor percentage of between 8 and 30% of an emulsifier with a high hydrophilic—lipophilic balance (HLB) and a major fraction of 70 to 92% of a second emulsifier having an HLB value of less than 10. Such compositions can lead to thermodynamically stable nano-emulsions formed by reversed micelles—hence sometimes also referred to as reverse solubilisates—in which the hydrophilic core holds the biologically active compound while both emulsifiers keep it stably dispersed in the continuous hydrophobic (oil) phase. In preferred embodiments the minor high-HLB emulsifier is a polysorbate or the vitamin E-derived surfactant tocopheryl polyethylene glycol succinate (TPGS). The major low-HLB emulsifier is preferably a sorbitan-ester with sorbitan oleate as the preferred excipient on which all the provided examples are based.

A disadvantage of the formulations of WO 2016/118907 is that the skin feel of compositions based on a stabilizing component with 70 to 92% of sorbitan oleate is poor. Furthermore, the formulations described in WO 2016/118907 are liquid also adding to the poor skin feel and which is detrimental for ease of handling and applying to the skin.

While WO 2016/118907 makes mention of “bee's wax” as an additional component to achieve higher viscosity, this does not automatically imply that the use of beeswax leads to formulations that are of acceptable semisolid consistency for the claimed compositions. As a matter of fact, it was found that the use of beeswax either made the compositions too hard and solid, or led to collapse of the semisolid consistency at lower percentages when the formulation is touched and applied.

Thus, there remains a need for improved skincare products that enable the creation of acceptable, agreeable and applicable (semi)solid oil-based skincare products with hydrophilic biologically active ingredient(s).

### BRIEF SUMMARY

Provided are compositions that overcome at least one or more of the disadvantages described herein above, in particular compositions that are semisolid or solid skincare products containing hydrophilic active ingredient(s) and that have an improved skin feel.

This disclosure therefore provides a composition comprising:
a continuous hydrophobic phase,
a hydrophilic phase dispersed in the hydrophobic phase,
a hydrophilic active ingredient dissolved in the hydrophilic phase, and
a stabilizing component comprising:
   4-20 wt % of an emulsifier having a hydrophilic-lipophilic balance (HLB) value of at least 10, and
   80-96 wt % of an emulsifier having an HLB value of less than 10,
wherein the continuous hydrophobic phase comprises a solid lipid component comprising 50-100 wt % of one or more high melting point triglycerides and between 0-50 wt % of one or more waxes.

In a further aspect, this disclosure provides a composition comprising:
a continuous hydrophobic phase,
a hydrophilic phase dispersed in the hydrophobic phase,
a hydrophilic active ingredient dissolved in the hydrophilic phase, and
a stabilizing component comprising:
   4-7.9 wt % of an emulsifier having a hydrophilic-lipophilic balance (HLB) value of at least 10, and
   92.1-96 wt % of an emulsifier having an HLB value of less than 10.

In a further aspect, this disclosure provides a composition comprising:
a continuous hydrophobic phase,
a hydrophilic phase dispersed in the hydrophobic phase,
a hydrophilic active ingredient dissolved in the hydrophilic phase, and
a stabilizing component comprising:
   4-20 wt % of an emulsifier having a hydrophilic-lipophilic balance (HLB) value of at least 10, and
   80-96 wt % of an emulsifier having an HLB value of less than 10,
wherein the weight ratio of the stabilizing component to the hydrophilic active ingredient is less than 10:1.

In a further aspect, this disclosure provides a method for the preparation of the composition according to any of the preceding claims, comprising:
dissolving the hydrophilic active substances in the hydrophilic phase,
dissolving the surfactant of high HLB value in the hydrophilic phase at elevated temperature,
gradually adding and mixing the surfactant of low HLB value in the same mixture while gradually cooling,
preparing the hydrophobic phase without the high melting point triglycerides and optionally the waxes by mixing the required ingredients,
mixing the hydrophobic phase into the mixture or mixing a large part of the hydrophobic phase into the mixture and dissolving the high melting point triglycerides and optionally the waxes in the remaining small part of the hydrophobic phase under heating to 60° C.,
heating the mixed formulation to 50-55° C.,
optionally mixing the solution of the high melting point triglycerides and optionally the waxes into the mixed formulation,
stirring and cooling the mixture to 50° C.,
pouring the mixture in the final containers.

The compositions are pharmaceutical or cosmetic compositions or formulations for topical administration, more in particular for administration to the skin. The compositions of this disclosure are thus preferably formulated for application on a body surface, preferably formulated for application on skin, scalp, nails, hair and mucosal membrane. Compositions of this disclosure can be, and are preferably, formulated as sticks, pastes, balms, ointments, unguents, salves, creams, gels and the more viscous types of lotions. In another embodiment, the compositions of this disclosure are formulated as a suppository, enema, adhesive bandage, patch or another topically applied health product.

### BRIEF DESCRIPTION OF THE DRAWINGS

FIG. 1: Depicted is a drop of formulation composed of a continuous hydrophobic phase including the solid lipid fraction (not separately indicated), which holds reverse micelles stabilized by a stabilizing component (spheres on the outer shell of the micelles) composed of the high and low HLB emulsifiers and functioning as an outer layer of the micelles. The inner core of the micelles holds the active molecules.

### DETAILED DESCRIPTION

The singular forms “a,” “an,” and “the” as used herein include plural referents unless the context clearly dictates otherwise. Thus, for example, reference to “a hydrophilic compound” includes reference to one or more of such compounds.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, the additional component(s) not altering the unique characteristic of this disclosure.

The word “approximately” or “about” when used in association with a numerical value (approximately 10, about 10) preferably means that the value may be the given value of 10 more or less 10% of the value.

By “pharmaceutically and/or cosmetically acceptable” it is meant that the carrier, diluent or excipient must be compatible with the other ingredients of the composition and not deleterious, e.g., toxic, to the recipient thereof.

The term “emulsion” refers to a mixture of two or more liquids wherein one liquid is present as dispersed droplets in another liquid in which it is not soluble or miscible. The emulsion may be a nanoemulsion. The dispersed droplets in emulsion may be in the form of reverse micelles. In this case the emulsion is often referred to as “reverse solubilisate,” which is thermodynamically stable and does not separate upon standing into a hydrophobic and a hydrophilic layer.

The term “reverse micelle” is used herein according to its art-recognized meaning, hence it refers to nanosized structures comprising a hydrophilic interior and a shell of surfactant molecules of which the hydrophilic (polar) part is facing the hydrophilic interior while the hydrophobic (apolar) part faces an exterior continuous oil phase in which the micelles are dispersed. The term includes all forms of reverse micelles, including, e.g., spherical reverse micelles, non-spherical reverse micelles, cylindrical reverse micelles, etc.

The term “stable” as used herein in connection with the compositions and formulations refers to compositions and formulations which exhibit no phase separation when kept, without agitation, at room temperature for one week or more.

The term “HLB” as used herein refers to a hydrophilic-lipophilic balance of a surface-active material (e.g., surfactant, emulsifier). An HLB value of 0 corresponds to a completely hydrophobic (i.e., lipophilic) molecule, and a value of 20 corresponds to a completely hydrophilic (i.e., lipophobic) molecule. As used herein a an emulsifier having an HLB value of at least 10 is referred to as a “high-HLB emulsifier” and an emulsifier having an HLB value of less than 10 is referred to as a “low-HLB emulsifier.”

As used herein, the term “substantially,” in reference to a given property or condition, refers to a degree that one of ordinary skill in the art would understand that the given property or condition is met with a small degree of variance, such as within acceptable measuring tolerances.

The term “high melting point triglycerides” as used herein refers to triglycerides that are solid at room temperature. In particular, it refers to an ester derived from glycerol and three fatty acids that is solid up to 40° C. or above. Such triglycerides usually consist of saturated long-chain fatty acids including but not limited to lauric, myristic, palmitic, stearic, arachidic and behenic acid.

The term “waxes” as used herein refers to fat-soluble substances with a melting point above 40° ° C. that can be pure long acyl-chain alkanes, long-acyl chain fatty acid esters of aliphatic primary or secondary alcohols, free fatty acids or free aliphatic alcohols both with long acyl chains. They can be of synthetic origin, of plant material, animal-derived or of petrochemical origin.

The term “solid” as used herein refers to solid at room temperature. In particular, it refers to having a melting point above 25° C.

The term “semisolid” as used herein is well known in the art. It refers to a state that resembles a solid (as opposed to a free-flowing fluid), but with the property of changing shape in response to force or pressure applied to it and/or having the ability to flow under pressure at room temperature. In cosmetics and skincare the term “semisolid” typically refers to pastes, balms, ointments, unguents, salves, creams, gels and the more viscous types of lotions, usually applied on skin or mucous membranes.

The term “hydrophilic” is used herein in relation to “phase” when is referred to a solvent or mixture of solvents that is not miscible with the oil phase, and can include alcohols, polyols, polyethylene glycols, and water. “Hydrophilic” is used herein in relation to “active ingredient(s)” or “compounds” or “substances” when is referred to material that is soluble in “hydrophilic phases” as defined above in at least a concentration of 10 mg/mL.

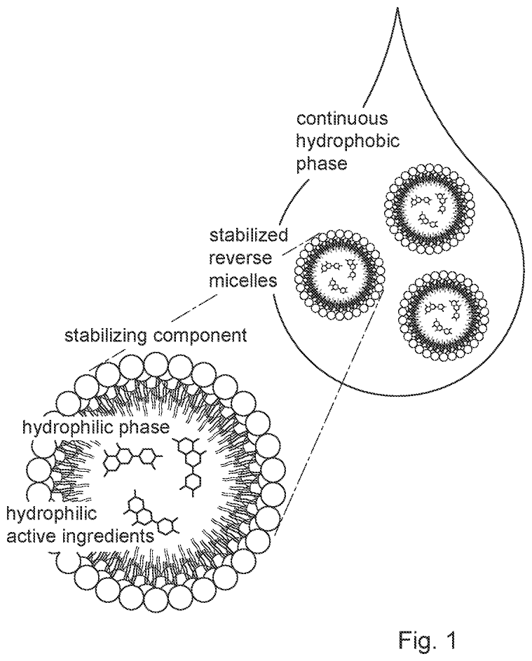

This disclosure relates to formulations for application on the human skin, wherein the formulation comprise a continuous hydrophobic phase, a hydrophilic phase, at least one hydrophilic biologically active compound substantially dissolved in the hydrophilic phase, and a stabilizing component composed of at least one emulsifier having an HLB value of at least 10 such as tocopheryl polyethylene glycol 1000 succinate (TPGS) and at least one emulsifier having an HLB value of less than 10, and which is characterized by having an excellent skin feel. A schematic representation of the compositions of this disclosure are shown in FIG. 1.

Compositions have been developed that feature a hydrophilic component dissolved in a hydrophilic phase that is stably formulated in a hydrophobic continuous phase in the form of reversed micelles enabled by a stabilizing component made of a minority fraction of high-HLB emulsifiers such as TPGS in combination with a majority fraction low-HLB emulsifiers with excellent skin feel, the formulations of which can be applied on the human skin as (semi)solid water-free/anhydrous skincare products. It was surprisingly discovered that this can be achieved by including a solid lipid fraction of which the majority is composed of one or more triglycerides that are solid at room temperature, with optionally a minority percentage of one or more waxes that are also solid at room temperature in the hydrophobic phase. This is surprising since WO 2016/118907 describes triglycerides as part of the “low-HLB emulsifier” but not as part the “continuous hydrophobic phase.” As a matter of fact, WO 2016/118907 teaches away from using the triglycerides by stating it should be absent in the majority of the particular embodiments described. Indeed, as a way to achieve higher viscosity WO 2016/118907 teaches the use of “bee's wax,” which either made the compositions too hard and solid, or led to collapse of the semisolid consistency at lower percentages when the formulation is touched and applied.

It has further been found that, to allow for the use of specific low-HLB emulsifiers for excellent skin feel, the stabilizing component of the skincare products preferably at least contain 92.1 weight % of this low-HLB emulsifier and at most 7.9 weight % of the high-HLB emulsifier. In the absence of the solid lipid, stable compositions can still be formulated with an excellent skin feel, although the compositions will have a more liquid and less solid form.

Moreover, it has been found that the compositions and formulations of this disclosure allow for a higher amount of hydrophilic active ingredient to be included in the hydrophilic phase than previously thought possible. Indeed, the weight ratio of the total stabilizing component to the hydrophilic component can be under 10:1 and can even be brought down to 5:1 or less in some cases, which can be considered a significant improvement. It is preferred that the total weight of the hydrophilic component is not more than 35 wt % of the total composition.

To enable the creation of a semisolid skincare product based on any of these compositions described above, it has further been discovered that between 7.5 and 20 weight % of the final composition is preferably composed of a mixture of triglycerides that are solid at room temperature, such as high melting point triglycerides, optionally including between 0 and 10 weight % of the final composition being one or more waxes, such as beeswax, beeswax alternative or other waxes in the same mixture as described below.

The presence of the solid lipid triglycerides and optionally waxes (preferably between 7.5 and 20 weight % of the final formulation) allows an end product consistency depending on the temperature at which it is allowed to solidify. The higher the temperature the more solid the final product will be.

Compositions comprising a continuous hydrophobic phase, a hydrophilic phase, at least one hydrophilic compound substantially dissolved in the hydrophilic phase, a stabilizing component are provided. The stabilizing component, for instance, comprises a minor fraction of tocopheryl polyethylene glycol 1000 succinate (TPGS) and a major fraction of a low HLB emulsifying agent. The composition is preferably stable for at least one week at room temperature without agitation. The composition is composed of stabilized reverse micelles of the hydrophilic compound(s) in the continuous hydrophobic phase.

A weight ratio of the stabilizing component to the hydrophilic active ingredient(s) in any of the compositions described herein is preferably below 10:1. The stabilizing component comprises the high-HLB emulsifier, e.g., TPGS, in an amount from about 4% to about 20% by weight based upon the total weight of the stabilizing component, and at least one emulsifier having an HLB value of less than 10 (preferably about 3 to 8, and more preferably about 3 to 6) present in the stabilizing component in an amount from about 80% to 96% by weight based upon the total weight of the stabilizing component.

In certain embodiments, the composition as described herein further comprises a mixture of triglycerides and waxes (preferably between 8 and 20% weight % of the final composition) to achieve a (semi)solid mass that can be applied on a human skin surface with excellent skin feel and acceptable end product consistency.

Continuous hydrophobic phases for use in reverse micelle compositions as the compositions of this disclosure are known to one of skill in the art. The continuous hydrophobic phase in any of the compositions provided herein constitutes between 20% and 60 wt % of the final formulation and preferably comprises pharmaceutically and/or cosmetically acceptable hydrophobic solvents, oils, butters, and waxes. They can include alkanes, mono-, di- and triglycerides, wax esters, fatty acids, silicon-based hydrophobic substances. They can be of natural origin, (partially) synthesized or from mineral sources. Often these substances are referred to as emollients, which highlights their occlusive nature on the skin, reducing the transepidermal water loss (TEWL). Examples of such hydrophobic solvents and oils may include, but are not limited to, alkanes varying from C6 to C100 (molecular mass between 80 and 1500) including squalane, squalene, hemisqualane, polyisobutene and liquid paraffin; monoalcohol (such as isopropanol or isoamyl alcohol) and polyol (such as glycerol) esters of saturated fatty acids such as capric, caprylic, lauric, myristic, palmitic, stearic, arachidic and behenic acid, or unsaturated fatty acids such as linoleic, linolenic, palmitoleic, oleic, docosahexaenoic, or eicosapentaenoic acid; triglyceride oils such as fractionated coconut oil, capric/caprylic triglyceride, jojoba, camellia seed, meadowfoam, canola, borage seed, castor, argan, sea buckthorn seed and pulp, safflower, corn, soybean, olive, cotton seed, sunflower seed, rice bran, oat bran, arachis, palm, rapeseed, hemp seed, pomegranate seed, calendula seed, chia seed, kiwi seed, raspberry seed, blueberry seed, strawberry seed, rosehip seed and linseed oil; butters including cocoa, mango, kokum, babassu, pracaxi, coconut and shea butter; silicon oils (often referred to as silicones) of which pentadimetylsiloxane (dimethicone) and decamethylcyclotetrasiloxane (cyclomethicone) are prime examples.

In certain embodiments the hydrophobic continuous phase comprises bioactive terpenes, notably monoterpenes and sesquiterpenes, that further add to the beneficial skin effects of the final formulations. Examples of such terpenes are carotol, humulene, beta-caryophyllene, bisabolol, chamazulene, nerolidol, terpinene-4-ol, farnesol and linalool. Diterpenes that can be mixed in the hydrophobic phase include retinoids and retinoid (vitamin A) derivatives. Tetraterpenes and tetraterpenoids including the carotenoids and xanthophylls can also be included in the hydrophobic phase.

In certain embodiments the hydrophobic continuous phase comprises bioactive fatty acid amides, such as palmitoylethanolamide, anandamide, or related bioactive structures.

If present, the solid lipid component is comprised in the continuous hydrophobic phase. In the hydrophobic phase preferably 10 to 45%, preferably 15 to 40%, by weight can be the solid lipid component, i.e., a mixture of high melting point triglycerides and waxes, to help create a (semi)solid mass that can be applied on the skin as a ointment, balm or salve. Examples of high melting point triglycerides are trimyristin, trilaurin, tripalmitin, tristearin, triarachidin and tribehenin. Preferred examples of waxes that can be used are beeswax, carnauba wax, candelilla wax, sunflower wax, rice bran wax, berry wax and myrica fruit wax. Other waxes that can be used are cetyl palmitate, cetyl alcohol and cetostearyl alcohol including their esters, and combinations thereof. Less preferred but also possible are the non-natural waxes ozokerite, paraffin, microcrystalline wax, ceresin, and petrolatum.

If present, the weight fraction of the mixture of high melting point triglycerides and waxes is preferably above 7.5 wt % of the final composition to ensure firmness at room temperature, while it preferably remains under 20 wt % of the final composition to retain sufficient softness for skin application. Preferably, the weight fraction is 10-15 wt % of the final composition. The final firmness and consistency of the formulation is dependent on the temperature at which it is allowed to solidify after mixing and processing. The addition of waxes to the mixture of high melting point triglycerides and waxes is believed to increase the strength of the formulations and can help make the formulations suitable e.g., for sticks. Preferably, between 5 and 35 wt % of the mixture of high melting point triglycerides and waxes is constituted by the waxes to create the stronger types of formulations.

Hydrophilic phases for use in reverse micelle compositions as the compositions of this disclosure are known to one of skill in the art. The hydrophilic phase in any of the compositions provided herein preferably comprises one or more hydrophilic solvents. Various non-toxic pharmaceutically and/or cosmetically acceptable hydrophilic solvents may be used to substantially dissolve the hydrophilic compound(s). Examples of such hydrophilic solvents include, but are not limited to, ethanol, dimethyl sulfoxide (DMSO), propylene glycol (PG), 1,3 propanediol, the different isomers of butanediol, glycerol, and polyethylene glycol (PEG) at different molecular weights (e.g., PEG 400 and PEG 800). These hydrophilic solvents can sometimes serve as humectants for the hydrophilic compounds, such as a pharmaceutically active ingredients, to be included in certain embodiments of the formulation. Thus, other humectants known to those having ordinary skill in the art can also be incorporated into the hydrophilic phase. The hydrophilic solvent may be present in the compositions in an amount sufficient to provide a stable dispersed phase of the hydrophilic compound in the continuous hydrophobic phase. Thus, for example, the fraction of hydrophilic solvent can vary from about 1 up to 25 wt % of the total composition. In preferred compositions the fraction of hydrophilic solvent is between 8 and 15 wt % of the final formulation.

The hydrophilic active ingredient in any of the compositions provided herein may comprise one or more substances that have a known or suspected biological activity in humans and that show sufficient solubility in the hydrophilic phase of at least 1% by weight.

The total amount of hydrophilic active ingredient(s) in the final formulation can be 2% by weight or higher, e.g., up to 7 or 8 wt %. The specific formulation of the compositions of this disclosure allow such high amounts of active ingredient. Hence, in one embodiment, the total amount of hydrophilic active ingredients in the final formulation is up to 10 wt %, e.g., 0.1-10 wt %. In another embodiment, the total amount of hydrophilic active ingredients in the final formulation is 2-10 wt %, more preferably 2-8 wt %.

In principle any hydrophilic active ingredient can be included in the compositions of this disclosure because the advantages described herein and resulting from are independent from the exact nature of the active ingredient. These advantage are obtained using the technology and specifically result from the specific combination of the components, in particular of the continuous hydrophobic phase, the hydrophilic phase, the stabilizing component and the solid lipid component.

In certain embodiments the hydrophilic active ingredient is of natural origin and derived from plant extracts. Examples of natural hydrophilic active ingredients from plant extracts include, but are not limited to, tea extracts and in particular purified polyphenols from these extracts such as epigallocatechin gallate (EGCG); grape extract and in particular purified (trans)-resveratrol; chamomile extract and in particular purified quercetin, apigenin, luteolin and chlorogenic acid and their glycosides; and other extracts that are particularly rich in flavonoid content from other plant sources such as bamboo extract, edelweiss extract, coffee extract, cocoa extract, cannabis extracts (isolates and distillates) and propolis.

In certain embodiments the hydrophilic active ingredient(s) are vitamins, including but not limited to vitamin C (ascorbic acid) and derivatives, and the different vitamins B and their derivatives.

In certain embodiments the hydrophilic active ingredient(s) are natural or synthetic pharmaceutical small molecular substances with biologic activity on or in the skin.

In certain embodiments the hydrophilic active ingredient(s) are peptides or peptide derivatives of natural or synthetic origin.

In certain embodiments the hydrophilic active ingredient(s) are prebiotic plant sugars or oligosaccharides such as xylitol, rhamnose, fructooligosaccharides including e.g., raspberry oligosaccharides, and glucomannan.

In certain embodiments the active ingredient(s) are minerals or derivatives thereof, of which orthosilicilic acid, its complexes and a derivative such as methylsilanetriol are a prime example.

The stabilizing component in any of the compositions provided herein comprises an emulsifier having a hydrophilic-lipophilic balance (HLB) value of at least 10, and an emulsifier having an HLB value of less than 10. The stabilizing component may comprise TPGS and/or at least one polysorbate emulsifier as the emulsifier having an HLB value of at least 10 in combination with at least one emulsifier having an HLB value of less than 10 (preferably from 3 to 8, and more preferably from 3 to 6). The weight ratio of the stabilizing component to the hydrophilic compound(s) in the final formulation is preferably at least about 5:1.

The emulsifier having an HLB value of less than 10 preferably has an HLB value from 3 to 8, and more preferably from 3 to 6.

In certain embodiments, the final formulation comprises the stabilizing component in an amount of at least about 10% by weight based upon total weight of the composition, more preferably at least 20 wt % based upon total weight of the composition.

In certain embodiments, a weight ratio of the continuous hydrophobic phase to the stabilizing component is no more than about 25:1. Thus, for example, the ratio can be from about 1:1, 2:1, 3:1, 4:1 or 5:1 to about 5:1, 6:1, 7:1, 8:1, 9:1, 10:1, 15:1, 20:1 or 25:1.

The emulsifier having an HLB value of at least 10, such as TPGS and/or a polysorbate emulsifier, preferably constitute about 4% to about 20% by weight of the stabilizing component, more preferably 4-7.9 wt %, more preferably 5-7.9 wt %. The emulsifier having an HLB value of less than 10 constitutes about 80% to about 96% by weight of the stabilizing component, more preferably 92.1-96 wt %, more preferably 92.1-95 wt %.

The final formulation preferably comprises no more than about 5% by weight of the emulsifier having an HLB value of at least 10, such as TPGS and/or a polysorbate emulsifier(s). The final formulation preferably comprises no more than about 60% by weight of the emulsifier having an HLB of less than 10.

TPGS is a water-soluble derivative of natural vitamin E, which is formed by esterification of vitamin E succinate with polyethylene glycol (PEG) 1000. TPGS is a nonionic surfactant having an HLB value of 13.2 and a relatively low critical micelle concentration (CMC) of 0.02% w/w. TPGS has an average molecular weight of 1,513 and completely dissolves in water. TPGS is an FDA approved pharmaceutically safe adjuvant, and its oral LD50 is reported to be more than 7 g/kg for young adult rats of both sexes. See, e.g., Guo et al., The Applications of Vitamin E TPGS in Drug Delivery, Eur. J. Pharmc. Sci. 49 (2013), 175-186.

Examples of the polysorbate emulsifiers that can be used in the stabilizing component are the PEG sorbitan fatty acid esters, which may include, but are not be limited to, PEG 20 sorbitan monolaurate (e.g., TWEEN® 20 from Croda Inc) PEG 20 sorbitan monopalmitate (e.g., TWEEN® 40 from Croda Inc.); PEG 20 sorbitan monostearate (e.g., TWEEN® 60 from Croda Inc); and PEG 20 sorbitan monooleate (e.g., polysorbate 80, TWEEN® 80 from Croda Inc).

The emulsifier having an HLB value of less than 10 (“low HLB emulsifier”) may include a cationic surfactant, an anionic surfactant, an amphoteric surfactant, a non-ionic surfactant, or a mixture thereof. Examples of low HLB emulsifiers are compounds selected from the group consisting of fatty acid mono-esters of glycerol, fatty acid di-esters of glycerol, fatty acid esters of polyglycerol, fatty acid mono-esters of propylene glycol, fatty acid di-esters of propylene glycol, fatty acid mono-esters of sorbitan, fatty acid di-esters of sorbitan, fatty acid tri-esters of sorbitan. Examples of fatty acid mono- and di-esters of glycerols may include, but are not be limited to, IMWITOR® 742 surfactant, which is a mixture of mono- and diglycerides of caprylic and capric acids from Sasol Olefins and Surfactants GmbH; IMWITOR® 988 surfactant, which is a mixture of mono- and diglycerides of caprylic acid from Sasol Olefins and Surfactants GmbH; IMWITOR 308 surfactant, which is a mixture of mono- and diglycerides of caprylic acid with more than about 80% monoglycerides from Sasol Olefins and Surfactants GmbH; IMWITOR 191, which is glyceryl monostearate from Huls AG/Huls America; CAPMUL MCM surfactant, which is monoglyceride of caprylic and capric acid from ABITEC Corporation; TEGIN OV surfactant, which is glyceryl monooleate from Evonik GmbH; CAPMUL GDL surfactant, which is glyceryl dilaurate from ABITEC Corporation; and MAISINE 35-1 surfactant, which is glyceryl monolinoleate from Gattefosse Corporation.

Examples of polyglycerol esters of fatty acids may include, but are not limited to, DERMOFEEL GO SOFT, which is polyglyceryl-2 sesquioleate from Evonik GmbH; ISOLAN GI 34, which is polyglyceryl-4 isostearate from Evonik GmbH; ISOLAN GO 33, which is polyglyceryl-3 Oleate from Evonik GmbH; ISOLAN GPS, which is polyglyceryl-4 diisostearate from Evonik GmbH; TEGO CARE PBS 6 MB, which is polyglyceryl-6 stearate and behenate from Evonik GmbH; TEGO SOLVE 90 MB, which is polyglyceryl-6 caprylate and caprate from Evonik GmbH; TEGOSOFT PC 31 MB, which is polyglyceryl-3 caprate from Evonik GmbH; and TEGOSOFT PC 41 MB, which is polyglyceryl-4 caprate form Evonik GmbH.

Examples of the fatty acid mono- and di-esters of propylene glycol may include, but are not be limited to, CAP TEX 200, which is propylene glycoldicaprylate/dicaprate from ABITEC Corporation; MIGLYO 840 surfactant, which is propylene glycol dicaprylate/dicaprate from Sasol Olefins and Surfactants GmbH; NEOBEE M-20 surfactant, which is propylene glycol dicaprylate/dicaprate from Stepan Company; LAUROGLYCOL surfactant, which is propylene glycol monolaurate from Gattefosse Corporation; MIRPYL surfactant, which is propylene glycol monolaurate; and CAPMULPG8 surfactant, which is propylene glycol monocaprylate with up to about 5% propylene glycol dicaprylate from ABITEC Corporation.

Examples of the fatty acid mono- and di-esters of sorbitan may include, but are not be limited to, sorbitan monooleate such as SPAN 80 surfactant from Croda Inc; sorbitan monopalmitate such as SPAN 40 surfactant from Croda Inc; sorbitan monolaurate such as SPAN 20 surfactant from Croda Inc; sorbitan monostearate such as SPAN 60 surfactant from Croda Inc; sorbitan sesquioleate such as SPAN 83 surfactant from Croda Inc; sorbitan monoisostearate such as SPAN 120 surfactant from Croda Inc; and sorbitan sesquistearate such as NIKKOL SS-15 surfactant from Nikkol Chemicals Co Ltd.

In preferred embodiments, the formulation includes a fatty acid ester of sorbitan which in particular contributes to an improved skin feel, particularly sorbitan sesquioleate, as at least one of the low HLB emulsifiers.

In certain embodiments, the formulation includes a polyglyceryl fatty acid ester as the low HLB emulsifier with excellent skin feel, particularly polyglyceryl-2-sesquioleate.

In certain embodiments, the formulation includes a glyceryl mono-ester as the low HLB emulsifier with excellent skin feel, particularly glyceryl monooleate.

Hence, in a preferred embodiment, the emulsifier having an HLB value of less than 10 comprises sorbitan sesquioleate, polyglyceryl-2-sesquioleate and/or glyceryl monooleate. In a particularly preferred embodiment, the emulsifier having an HLB value of less than 10 is sorbitan sesquioleate, polyglyceryl-2-sesquioleate and/or glyceryl monooleate. If sorbitan sesquioleate, polyglyceryl-2-sesquioleate and/or glyceryl monooleate are chosen as the stabilizing component then preferably at least 50 wt % of the emulsifier having an HLB value of less than 10 is sorbitan sesquioleate, polyglyceryl-2-sesquioleate and/or glyceryl monooleate, more preferably 100% of the emulsifier having an HLB value of less than 10 is sorbitan sesquioleate, polyglyceryl-2-sesquioleate and/or glyceryl monooleate. In certain embodiments, the stabilizing component in the formulation comprises a TPGS emulsifier and at least one low HLB emulsifier selected from sorbitan sesquioleate, polyglyceryl-2-sesquioleate, glyceryl monooleate and combinations thereof.

The compositions that can be made according to this disclosure are (semi)solid formulations that can be applied on body surfaces including the skin, scalp, nails, hair and mucosal membranes. Hence, in a preferred embodiment, the compositions of this disclosure are formulated for application on a body surface, preferably formulated for application on skin, scalp, nails, hair and mucosal membrane. Compositions of this disclosure can be, and are preferably, formulated as sticks, pastes, balms, ointments, unguents, salves, creams, gels and the more viscous types of lotions. Other potential ways this disclosure can be applied include suppositories, enemas, adhesive bandages, patches and other topically applied health products.

In general, the process for the preparation of the compositions according to this disclosure may comprise:
dissolving the hydrophilic active substances in the hydrophilic phase;
dissolving the surfactant of high HLB value in the hydrophilic phase at elevated temperature;
gradually adding and mixing the surfactant of low HLB value in the same mixture while gradually cooling;
preparing the hydrophobic phase without the high melting point triglycerides and optionally the waxes by mixing the required ingredients;
mixing a large part of the hydrophobic phase into the mixture or dissolving the high melting point triglycerides and optionally the waxes in the remaining small part of the hydrophobic phase under heating to 65° C.;
heating the mixed formulation to 50-55° C.;
optionally mixing the solution of the high melting point triglycerides and optionally the waxes into the mixed formulation;
stirring and cooling to 50° C.; stirring can be done with mixing aids, whipping aids or whisks to further improve the consistency of the formulation; and
pouring the mixture in the final containers at a desired temperature below 50° C.

Optionally the procedure is performed under a protective atmosphere of nitrogen, argon and/or other noble gases.

Features may be described herein as part of the same or separate aspects or embodiments of this disclosure for the purpose of clarity and a concise description. It will be appreciated by the skilled person that the scope of this disclosure may include embodiments having combinations of all or some of the features described herein as part of the same or separate embodiments.

This disclosure will be explained in more detail in the following, non-limiting examples.

### EXAMPLES

The following formulation Examples were prepared as described below. Example 6 is a comparative Example in which a formulation as described in WO 2016/118907 was prepared.

The formulations were evaluated blindly by a test panel. The formulations were scored on a scale of 1-5 stars (with 1 being the lowest score and 5 being the highest score) for consistency/texture, spreadability, absence of tackiness, smell and afterfeel. These 5 scores were averaged into a single score for skin feel for each member of the test panel. Mean skin feel scores of all members of the test panel are indicated in the Examples.

### Example 1

Formulation comprising hydrophilic EGCG, chlorogenic acid, resveratrol, tea polyphenols and quercetin in a continuous hydrophobic phase comprising hemisqualane, squalane, babassu oil, mango butter, beeswax, tribehenin and sunflower wax.

The hydrophilic epigallocatechin gallate (EGCG catechins) (tea extract) Sunphenon from Lithos Ingredients BV was selected together with resveratrol from Lithos Ingredients and chlorogenic acid, tea polyphenols and quercetin from NanJing Manhay Medical Technology Inc. Propylene glycol (PG) was used as the hydrophilic solvent. Squalane and hemisqualane (Safic-Alcan, Benelux NV/SA) were used as continuous hydrophobic phase to which babassu oil and mango butter (both Croda GmbH) were added. TPGS was obtained from Gustav Parmentier GmbH and used as an emulsifier having a hydrophilic-lipophilic balance (HLB) value of at least 10, while sorbitan sesquioleate (Croda GmbH) was used as an emulsifier having an HLB value of less than 10. Tribehenin (Croda GmbH), beeswax (TKB Trading Inc), and sunflower wax (MakingCosmetics Inc) were used as the solid lipid fraction.

EGCG, chlorogenic acid, resveratrol, tea polyphenols and quercetin (in a ratio 2:1:0.5:0.5:0.5) were dissolved in propylene glycol (PG) hydrophilic solvent to produce a solution (the hydrophilic phase) having a weight ratio of actives to PG solvent of about 4.5:8. The solution of actives was subsequently mixed with the stabilizing component, of which a fraction of 5.5 weight % was TPGS and 94.5 weight % sorbitan sesquioleate emulsifier (weight ratio stabilizing component to hydrophilic active ingredients is approximately 9:1), forming approximately 53 weight % of the final formulation, to which then an equal mixture of squalane, hemisqualane, babassu oil and mango butter was added as the hydrophobic continuous fluid fraction forming approximately 32 weight % of the final formulation, followed (upon heating to 55 degrees C.) by a mixture of melted beeswax, tribehenin and sunflower wax (weight ratio 1:2:0.3) as the solid lipid fraction forming 15 weight % of the final formulation.

The resulting mixture contains around 4.5 weight % hydrophilic active ingredients and has an agreeable balm texture depending on the temperature at which it is poured into the final container and an excellent skin feel (mean skin feel score of five users: 4.4 out of 5 stars).

### Example 2

Formulation comprising hydrophilic luteolin, chlorogenic acid, apigenin and quercetin in a continuous hydrophobic phase comprising hemisqualane, squalane, babassu oil, mango butter, beeswax, trilaurin, tripalmitin and cetyl palmitate.

The hydrophilic active ingredients luteolin, chlorogenic acid, apigenin and quercetin (NanJing Manhay Medical Technology Inc) were mixed in a ratio 1:0.5:1:0.5 and dissolved in PEG 400 (Fagron Belgium NV) in a weight ratio of actives to PEG solvent of about 1:8 forming the hydrophilic phase. The hydrophilic phase was subsequently mixed with the stabilizing component, which was composed of 7 weight % TPGS and 93 weight % polyglyceryl-2 sesquioleate (weight ratio stabilizing component to hydrophilic active ingredients 30:1), forming approximately 56 weight % of the final formulation, to which then an equal mixture of squalane, hemisqualane and babassu oil was added as the hydrophobic continuous fluid fraction forming approximately 32.5 weight % of the final formulation followed (upon heating to 55 degrees C.) by a mixture of melted beeswax, trilaurin, tripalmitin (both TCI Inc.) and cetyl palmitate (weight ratio 1:0.5:0.5:0.3) as the solid lipid fraction forming 11.5 weight % of the final formulation. To the final formulation 1% of German chamomile CO2 extract was added. The formulation has an agreeable salve consistency depending on the pouring temperature and greenish color. It contains around 1.5 weight % hydrophilic active ingredients and has an excellent skin feel (mean skin feel score of five users: 4.2 out of 5 stars).

### Example 3

Formulation comprising hydrophilic EGCG, chlorogenic acid and vitamin C (ascorbic acid) in a continuous hydrophobic phase comprising hemisqualane, squalane, babassu oil and tribehenin (with no further waxes added).

The hydrophilic epigallocatechin gallate (EGCG catechins) (tea extract) Sunphenon from Lithos Ingredients BV was selected together with ascorbic acid from Fagron Belgium NV and chlorogenic acid from NanJing Manhay Medical Technology Inc.

EGCG, chlorogenic acid, and vitamin C (in a ratio 2:1:2) were dissolved in propylene glycol (PG) hydrophilic solvent to produce a solution (the hydrophilic phase) having a weight ratio of actives to PG solvent of 7.5:14. The solution of actives was subsequently mixed with the stabilizing component, of which a fraction of 10.5 weight % was TPGS and 89.5 weight % glyceryl monooleate emulsifier (weight ratio stabilizing component to hydrophilic active ingredients was around 4.5:1), forming approximately 52 weight % of the final formulation, to which then an equal mixture of squalane, hemisqualane and babassu oil was added as the hydrophobic continuous fluid fraction forming approximately 34.5 weight % of the final formulation, followed (upon heating to 55 degrees C.) by melted tribehenin as the solid lipid fraction forming 13.5 weight % of the final formulation.

The resulting mixture contains around 7.5 weight % hydrophilic active ingredients. The final product has a slight pink color an agreeable creamy texture depending on the temperature at which it is poured into the final container and an excellent skin feel (mean skin feel score of five users: 4.2 out of 5 stars).

### Example 4

Sunscreen formulation comprising hydrophilic luteolin, chlorogenic acid and resveratrol in a continuous hydrophobic phase comprising hemisqualane, squalane, babassu oil, mango butter, beeswax, tribehenin and cetyl palmitate.

The hydrophilic active ingredients luteolin, chlorogenic acid and resveratrol (NanJing Manhay Medical Technology Inc) were mixed in a ratio 1.5:2.5:1.5 and dissolved in PEG 400 (Fagron Belgium NV) in a weight ratio of actives to PEG solvent of about 5.5:14 forming the hydrophilic phase. The hydrophilic phase was subsequently mixed with the stabilizing component, of which a fraction of 10.5 weight % was TPGS and 89.5 weight % glyceryl monooleate emulsifier (weight ratio stabilizing component to hydrophilic active ingredients was around 6:1), forming approximately 53.5 weight % of the final formulation, to which then an equal mixture of squalane, hemisqualane and babassu oil was added as the hydrophobic continuous fluid fraction forming approximately 31.5 weight % of the final formulation followed (upon heating to 55 degrees C.) by a mixture of melted beeswax, tribehenin, sunflower wax and cetyl palmitate (weight ratio 1:1:07:0.3) as the solid lipid fraction forming 15 weight % of the final formulation. The formulation has an agreeable salve consistency depending on the pouring temperature and an off-white color. It contains around 5.5 weight % hydrophilic active ingredients and has an excellent skin feel (mean skin feel score of five users: 4.0 out of 5 stars). Furthermore, in a human skin test involving healthy volunteers it has an Sun Protection Factor (SPF) of around 10.

### Example 5

Formulation comprising hydrophilic EGCG, chlorogenic acid, bamboo leaf flavonoids and vitamin C (ascorbic acid) in a continuous hydrophobic phase comprising hemisqualane, squalane, babassu oil, beeswax and tribehenin to which vitamin E and vitamin A palmitate are added.

The hydrophilic epigallocatechin gallate (EGCG catechins) (tea extract) Sunphenon from Lithos Ingredients BV was selected together with ascorbic acid from Fagron Belgium NV and bamboo leaf flavonoids and chlorogenic acid from NanJing Manhay Medical Technology Inc.

EGCG, resveratrol, chlorogenic acid, bamboo leaf flavonoids and vitamin C (in a ratio 1.5:0.5:1:0.5:1) were dissolved in propylene glycol (PG) hydrophilic solvent to produce a solution (the hydrophilic phase) having a weight ratio of actives to PG solvent of 4.5:10. The solution of actives was subsequently mixed with the stabilizing component, of which a fraction of 10.5 weight % was TPGS and 89.5 weight % glyceryl monooleate emulsifier (weight ratio stabilizing component to hydrophilic active ingredients was around 5:1), forming approximately 39.5 weight % of the final formulation, to which then an equal mixture of squalane, hemisqualane and babassu oil was added as the hydrophobic continuous fluid fraction forming approximately 45.5 weight % of the final formulation, followed (upon heating to 55 degrees C.) by melted tribehenin as the solid lipid fraction forming 13.5 weight % of the final formulation. To the final formulation also 1 weight % vitamin E (tocopherol) and 0.5 weight % vitamin A (as retinyl palmitate) was added.

The resulting mixture contains around 4.5 weight % hydrophilic active ingredients and 1.5% hydrophobic active ingredients. The final product has a slight beige color an agreeable creamy texture depending on the temperature at which it is poured into the final container and an excellent skin feel.

### Comparative Example 6

Formulation comprising hydrophilic apigenin and quercetin in a continuous hydrophobic phase comprising hemisqualane, squalane, babassu oil, mango butter, beeswax and paraffin.

The hydrophilic active ingredients apigenin and quercetin (NanJing Manhay Medical Technology Inc) were mixed in a ratio 1:0.5 and dissolved in PEG 400 (Fagron Belgium NV) in a weight ratio of actives to PEG solvent of about 1:15 forming the hydrophilic phase. The hydrophilic phase was subsequently mixed with the stabilizing component, which was composed of 11.5 weight % TPGS and 88.5 weight sorbitan oleate (weight ratio stabilizing component to hydrophilic active ingredients 35:1), forming approximately 32 weight % of the final formulation, to which then an equal mixture of squalane, hemisqualane and babassu oil was added as the hydrophobic continuous fluid fraction forming approximately 58 weight % of the final formulation followed (upon heating to 55 degrees C.) by a mixture of melted beeswax and paraffin (weight ratio 1:1) without triglycerides as the solid lipid fraction forming 9 weight % of the final formulation. To the final formulation 1% of German chamomile CO2 extract was added. The formulation has a poor texture and a tacky skin feel (mean skin feel score of five users: 2.8 out of 5 stars). It contains around 0.75 weight % hydrophilic active ingredients.

1. A composition comprising:
a continuous hydrophobic phase,
a hydrophilic phase dispersed in said hydrophobic phase,
a hydrophilic active ingredient dissolved in the hydrophilic phase, and
a stabilizing component comprising:
4-20 wt % of an emulsifier having a hydrophilic-lipophilic balance (HLB) value of at least 10, and
80-96 wt % of an emulsifier having an HLB value of less than 10,
wherein the continuous hydrophobic phase comprises a solid lipid component comprising 50-100 wt % of one or more high melting point triglycerides and between 0-50 wt % of one or more waxes.

2. A composition comprising:
a continuous hydrophobic phase,
a hydrophilic phase dispersed in said hydrophobic phase,
a hydrophilic active ingredient dissolved in the hydrophilic phase, and
a stabilizing component comprising:
4-7.9 wt % of an emulsifier having a hydrophilic-lipophilic balance (HLB) value of at least 10, and
92.1-96 wt % of an emulsifier having an HLB value of less than 10.

3. A composition comprising:
a continuous hydrophobic phase,
a hydrophilic phase dispersed in said hydrophobic phase,
a hydrophilic active ingredient dissolved in the hydrophilic phase, and
a stabilizing component comprising:
4-20 wt % of an emulsifier having a hydrophilic-lipophilic balance (HLB) value of at least 10, and
80-96 wt % of an emulsifier having an HLB value of less than 10,
wherein the weight ratio of the stabilizing component to the hydrophilic active ingredient is less than 10:1.

4. The composition of claim 3, wherein the continuous hydrophobic phase comprises a solid lipid component comprising 50-100 wt % of one or more high melting point triglycerides and 0-50% of one or more waxes.

5. The composition according to claim 1, wherein the solid lipid component is present in an amount of 10-20% based on the total weight of the composition.

6. The composition according to claim 1, wherein the triglyceride is selected from the group consisting of trilaurin, tripalmitin, tristearin, triarachidin and tribehenin.

7. The composition according to claim 1, wherein the triglyceride is tribehenin.

8. The composition of claim 1, wherein the stabilizing component comprises 5-7.9% of the emulsifier having an HLB value of at least 10 and 92.1-95% of the emulsifier having an HLB of less than 10.

9. The composition of claim 1, wherein the emulsifier having an HLB value of less than 10 comprises a fatty acid mono-ester of sorbitan.

10. The composition of claim 1, wherein the emulsifier having an HLB value of less than 10 is sorbitan sesquioleate, polyglyceryl-2-sesquioleate and/or glyceryl monooleate.

11. The composition of claim 1, wherein the emulsifier having an HLB value of at least 10 comprises D-a-tocopheryl polyethylene glycol 1000 succinate (TPGS).

12. The composition of claim 1, wherein the emulsifier having an HLB value of less than 10 is sorbitan sesquioleate, polyglyceryl-2-sesquioleate and/or glyceryl monooleate.

13. The composition of claim 1, wherein the weight ratio of the stabilizing component to the hydrophilic active ingredient is less than 10:1.

14. The composition of claim 1, wherein the stabilizing component comprises 5-6.9 wt % of TPGS and 92.1-95 wt % sorbitan sesquioleate, polyglyceryl-2-sesquioleate and/or glyceryl monooleate.

15. A method for preparing the composition of claim 1, comprising:
dissolving the hydrophilic active substances in the hydrophilic phase,
dissolving the surfactant of high HLB value in the hydrophilic phase at elevated temperature,
gradually adding and mixing the surfactant of low HLB value in the same mixture while gradually cooling,
preparing the hydrophobic phase without the high melting point triglycerides and the waxes by mixing the required ingredients,
mixing the hydrophobic phase into the mixture or mixing a large part of the hydrophobic phase into the mixture and dissolving the high melting point triglycerides and optionally the waxes in the remaining small part of the hydrophobic phase under heating to 60° C.,
heating the mixed formulation to 50-55° C.,
optionally mixing the solution of the high melting point triglycerides and optionally the waxes into the mixed formulation,
stirring and cooling the mixture to 50° C., and
pouring the mixture in final containers.

16. The composition of claim 3, wherein the stabilizing component comprises 5-7.9% of the emulsifier having an HLB value of at least 10 and 92.1-95% of the emulsifier having an HLB of less than 10.

17. The composition of claim 3, wherein the emulsifier having an HLB value of less than 10 comprises a fatty acid mono-ester of sorbitan.

18. The composition of claim 3, wherein the emulsifier having an HLB value of less than 10 is sorbitan sesquioleate, polyglyceryl-2-sesquioleate and/or glyceryl monooleate.

19. The composition of claim 3, wherein the emulsifier having an HLB value of at least 10 comprises D-a-tocopheryl polyethylene glycol 1000 succinate (TPGS).

20. The composition of claim 3, wherein the emulsifier having an HLB value of less than 10 is sorbitan sesquioleate, polyglyceryl-2-sesquioleate and/or glyceryl monooleate.
